(19) United States
(12) Patent Application Publication
(10) Pub. No.: US 2004/0052743 A1
(43) Pub. Date: Mar. 18, 2004

(54) Five-layered pigments

(75) Inventors: Christoph Schmidt, Kriftel (DE); Andrea Heyland, Reichelsheim (DE); Sabine Schoen, Herten (DE); Veronika Hochstein, Bruchsal (DE); Uta Hensen, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH, Darmstadt (DE)

(21) Appl. No.: 10/608,563

(22) Filed: Jun. 30, 2003

(30) Foreign Application Priority Data
Jun. 28, 2002 (DE) 102 29 256.6
Nov. 1, 2002 (DE) 102 51 378.3

(51) Int. Cl. A61K 7/021; C09C 1/36; C04B 2/00

(52) U.S. Cl. 424/63; 106/436

(57) ABSTRACT

The present invention relates to five-layered pigments based on multi-coated platelet-shaped substrates which comprise a layer sequence comprising

(A) a layer of SnO₂ having a layer thickness of 0.1-50 nm,

(B) a high-refractive-index coating consisting of TiO₂ in the rutile modification having a layer thickness of 10-800 nm,

(C) a colorless coating having a refractive index n≦1.8 having a layer thickness of 20-800 nm,

(D) a high-refractive-index coating consisting of SnO₂ having a layer thickness of 0.1-50 nm,

(E) a layer of TiO₂ in the rutile modification having a layer thickness of 10-800 nm,

and optionally

(F) an outer protective layer,

and to the use thereof in paints, coatings, powder coatings, printing inks, security printing inks, plastics, ceramic materials, glasses, as dopants for the laser marking of papers and plastics, in cosmetic formulations and for the preparation of pigment preparations and dry preparations.

The present invention relates to five-layered pigments based on multi-coated platelet-shaped substrates.

Luster or effect pigments are employed in many areas of industry, in particular in the area of automotive finishes, decorative coatings, plastics, paints, printing inks and in cosmetic formulations.

Owing to their color play, luster pigments, which exhibit an angle-dependent color change between a plurality of interference colors, are of particular interest for automotive finishes and in forgery-proof documents of value.

The prior art discloses processes for the preparation of pearlescent pigments with the aid of which alternating layers of high and low refractive index can be applied to finely divided substrates. Pigments of this type based on multicoated platelet-shaped substrates are disclosed, for example, in U.S. Pat. No. 4,434,010, JP H7-759, U.S. Pat. No. 3,438,796, U.S. Pat. No. 5,135,812, DE 44 05 494, DE 44 37 753, DE 195 16 181 and DE 195 15 988, DE 196 18 565, DE 197 46 067 and in the literature, for example in EURO COSMETICS, 1999, No. 8, p. 284.

Mineral-based pearlescent pigments are of particular importance. Pearlescent pigments are prepared by coating an inorganic, platelet-shaped support with a high-refractive-index, usually oxidic layer. The color of these pigments is caused by wavelength-selective partial reflection and interference of the reflected or transmitted light at the medium/oxide or oxide/substrate interfaces.

The interference color of these pigments is determined by the thickness of the oxide layer. The hue of a silver interference pigment is generated by an (in the optical sense) single high-refractive-index layer whose optical thickness causes a reflection maximum (1 st order) in the visible wave-length range at about 500 nm. This wavelength is perceived by the human eye as the color green. However, the intensity curve of this maximum on its wavelength axis is so broad that so much light is reflected throughout the region of visible light that the human eye perceives a very bright, but colorless impression.

According to the rules known—in particular from the coating of optical components—for the optics of third layers, the intensity at the interference maximum increases by more than 60% compared with the single-layer system. Accordingly, the profile of the light reflected by interference is significantly more pronounced, meaning that a green reflection color can be expected for a multilayered system of this type.

Surprisingly, five-layered pigments have now been found which, with respect to their coloristic, but also applicational properties, exhibit significantly improved properties compared with the multilayered pigments from the prior art. This has been achieved by internal structuring of the high-refractive-index layers.

The invention therefore relates to an at least five-layered pigment comprising a platelet-shaped substrate which comprises thereon at least a layer sequence of

(A) a layer of SnO₂, preferably having a layer thickness of 0.1-50 nm,

(B) a layer of TiO₂ in the rutile modification preferably having a layer thickness of 10-800 nm,

(C) a colorless coating having a refractive index n≦1.8 preferably having a layer thickness of 20-800 nm,

(D) a layer of SnO₂ preferably having a layer thickness of 0.1-50 nm,

(E) a layer of TiO₂ in the rutile modification preferably having a layer thickness of 10-800 nm,

and optionally

(F) an outer protective layer.

The multilayered pigments according to the invention are interference pigments which, compared with the known multilayered pigments having three layers, have

significantly increased brightness

greater luster and

a more pronounced color flop.

The invention furthermore relates to the use of the multilayered pigments according to the invention in paints, coatings, powder coatings, printing inks, plastics, ceramic materials, glasses and cosmetic formulations, in particular in decorative cosmetics. The pigments according to the invention are furthermore also suitable for the preparation of pigment preparations and for the preparation of dry preparations, such as, for example, granules, chips, pellets, briquettes, etc. The dry preparations are particularly suitable for printing inks.

Suitable base substrates for the multilayered pigments according to the invention are colorless or selectively or nonselectively absorbent platelet-shaped substrates. Preferred substrates are phyllosilicates. Particularly suitable are natural and/or synthetic mica, talc, kaolin, platelet-shaped iron or aluminium oxides, glass, SiO₂, TiO₂ and graphite platelets, synthetic support-free platelets, titanium nitride, titanium silicide, liquid crystal polymers (LCPs), holographic pigments, BiOCl, platelet-shaped mixed oxides, such as, for example, FeTiO₃ or Fe₂TiO₅, or other comparable materials.

The size of the base substrates is not crucial per se and can be matched to the particular application. In general, the platelet-shaped substrates have a thickness of between 0.005 and 10 μm, in particular between 0.05 and 5 μm. The size in the other two dimensions is usually between 1 and 500 μm, preferably between 2 and 200 μm and in particular between 5 and 60 μm.

The thickness of layer (A) or (D) is 0.1-50 nm, preferably 0.3-30 nm, in particular 0.5-20 nm. The SnO₂ layers (A) and (D) can have identical or different layer thicknesses.

The thickness of the individual layers (B) and (C) and (E) of high or low refractive index on the base substrate is essential for the optical properties of the pigment. For the multilayered pigment according to the invention, the thicknesses of the individual layers must be set precisely with respect to one another.

The thickness of layer (B) or (E) is 10-800 nm, preferably 20-500 nm, in particular 30-400 nm. The TiO₂ layers (B) and (E) can have identical or different layer thicknesses. The thickness of layer (C) is 20-800 nm, preferably 30-600 nm, in particular 40-500 nm.

Colorless, low-refractive-index materials which are suitable for the coating (C) are preferably metal oxides or the corresponding oxide hydrates, such as, for example, SiO₂, Al₂O₃, AlO(OH), B₂O₃, MgF₂, MgSiO₃ or a mixture of the said metal oxides. Layer (C) is particularly preferably an SiO₂ layer.

The five-layered pigments according to the invention can be prepared by forming a plurality of high- and low-refractive-index interference layers having a precisely defined thickness and a smooth surface on the finely divided, platelet-shaped substrates.

The metal-oxide layers are preferably applied by wet-chemical methods, it being possible to use the wet-chemical coating methods developed for the preparation of pearlescent pigments. Methods of this type are described, for example, in DE 14 67 468, DE 19 59 988, DE 20 09 566, DE 22 14 545, DE 22 15 191, DE 22 44 298, DE 23 13 331, DE 15 22 572, DE 31 37 808, DE 31 37 809, DE 31 51 343, DE 31 51 354, DE 31 51 355, DE 32 11 602, DE 32 35 017, and also in further patent documents and other publications known to the person skilled in the art.

In the case of wet coating, the substrate particles are suspended in water, and one or more hydrolyzable metal salts or a water-glass solution are added at a pH which is suitable for hydrolysis, the latter being selected in such a way that the metal oxides or metal oxide hydrates are precipitated directly onto the platelets without secondary precipitations occurring. The pH is usually kept constant by simultaneous metering-in of a base and/or acid. The pigments are subsequently separated off, washed and dried, e.g., at 50-150° C. for 6-18 hours and, if desired, calcined, e.g., for 0.5-3 hours, where the calcination temperature can be optimized with respect to the coating present in each case. In general, the calcination temperatures are between 250 and 100° C., preferably between 350 and 900° C. If desired, the pigments can be separated off, dried and, if desired, calcined after application of individual coatings and then re-suspended for precipitation of the further layers.

The coating can furthermore also be carried out in a fluidized-bed reactor by gas-phase coating, it being possible, for example, correspondingly to use the processes proposed in EP 0 045 851 and EP 0 106 235 for the preparation of pearlescent pigments.

The hue of the interference pigments can be varied in very broad limits by choosing different covering amounts or layer thicknesses resulting therefrom. The fine tuning for a certain hue can be achieved beyond the pure choice of amount by approaching the desired color under visual or measurement technology control.

In order to increase the light, water and weather stability, it is frequently advisable, depending on the area of application, to subject the finished pigment to post-coating or post-treatment. Suitable post-coatings or post-treatments are the processes described, for example, in German Patent 22 15 191, DE-A 31 51 354, DE-A 32 35 017 and DE-A 33 34 598. This post-coating (layer F) further increases the chemical stability or simplifies handling of the pigment, in particular incorporation into various media.

The pigments according to the invention are compatible with a multiplicity of color systems, preferably from the area of paints, coatings and printing inks. For the production of printing inks for, for example, gravure printing, flexographic printing, offset printing or offset overprinting, a multiplicity of binders, in particular water-soluble grades, as marketed, for example, by BASF, Marabu, Pröll, Sericol, Hartmann, Gebr. Schmidt, Sicpa, Aarberg, Siegberg, GSB-Wahl, Follmann, Ruco or Coates Screen INKS GmbH, are suitable. The printing inks may be water-based or solvent-based. The pigments are furthermore also suitable for the laser marking of paper and plastics and for applications in the agricultural sector, for example for greenhouse sheeting, and, for example, for the coloring of tent awnings.

Since the multilayered pigments according to the invention combine high luster with high brightness, transparency and a very pronounced color flop, particularly useful effects can be achieved therewith in the various application media, for example in cosmetic formulations, such as, for example, nail varnishes, lipsticks, compact powders, gels, lotions, soaps and toothpastes, in paints, such as, for example, automotive finishes, industrial coatings and powder coatings, and in plastics and in ceramics.

Owing to the good skin feeling and very good skin adhesion, the pigments according to the invention are particularly suitable for decorative cosmetics.

For the various applications, the multilayered pigments according to the invention can also advantageously be used in the form of a mixture with organic dyes, organic pigments or other pigments, such as, for example, transparent and opaque white, colored and black pigments, and with platelet-shaped iron oxides, organic pigments, holographic pigments, LCPs (liquid crystal polymers) and conventional transparent, colored and black luster pigments based on metal oxide-coated mica and SiO₂ platelets, etc. The five-layered pigments according to the invention can be mixed with commercially available pigments and fillers in any ratio.

Fillers which may be mentioned are, for example, natural and synthetic mica, nylon powder, pure or filled melamine resins, talc, glasses, kaolin, oxides or hydroxides of aluminium, magnesium, calcium, zinc, BiOCl, barium sulfate, calcium sulfate, calcium carbonate, magnesium carbonate, carbon, and physical or chemical combinations of these substances.

There are no restrictions regarding the particle shape of the filler. It can be, for example, platelet-shaped, spherical or needle-shaped in accordance with requirements.

In the formulations, the pigments according to the invention can of course also be combined with cosmetic raw materials and auxiliaries of all types. These include, inter alia, oils, fats, waxes, film formers, preservatives and auxiliaries which generally determine applicational properties, such as, for example, thickeners and rheological additives, such as, for example, bentonites, hectorites, silicon dioxides, Ca silicates, gelatin, high-molecular-weight carbohydrates and/or surface-active auxiliaries, etc.

The formulations comprising the pigments according to the invention can belong to the lipophilic, hydrophilic or hydrophobic type. In the case of heterogeneous formulations having:discrete aqueous and non-aqueous phases, the pigments according to the invention can each be present in only one of the two phases or alternatively distributed over the two phases.

The pH values of the formulations can be between 1 and 14, preferably between 2 and 11 and particularly preferably between 5 and 8.

No limits are set on the concentrations of the pigments according to the invention in the formulation. These concentrations can—depending on the application—be between 0.001 (rinse-off products, for example shower gels) and 100% (for example luster-effect articles for particular applications).

The pigments according to the invention may furthermore also be combined with cosmetic active-ingredients. Suitable active ingredients are, for example, insect repellents, UV A/BC protection filters (for example OMC, B3 and MBC), anti-ageing active ingredients, vitamins and derivatives thereof (for example vitamin A, C, E, etc.), self-tanning agents (for example DHA, erythrulose, inter alia), and further cosmetic active ingredients, such as, for example, bisabolol, LPO, ectoin, emblica, allantoin, bioflavonoids and derivatives thereof.

The pigments according to the invention are furthermore suitable for the preparation of flowable pigment preparations and dry preparations comprising one or more pigments according to the invention, binders and optionally one or more additives. The term dry preparations is also taken to mean preparations which comprise from 0 to 8% by weight of water and/or a solvent or solvent mixture. The dry preparations are preferably in the form of pellets, granules, chips and briquettes. The dry preparations are preferably employed in the production of printing inks and in cosmetic formulations.

The invention furthermore also relates to the use of the pigments in formulations such as paints, printing inks, security printing inks, coatings, plastics, ceramic materials, glasses, in cosmetic formulations, as dopants for the laser marking of papers and plastics and for the preparation of pigment preparations and dry preparations.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLES

### Example 1

5-Layered Pigment Having a Pronounced Color Flop

100 g of mica having a particle size of 10-60 μm are heated to 80° C. in 2 l of demineralised water with stirring. When this temperature has been reached, a solution consisting of 2.3 g of SnCl₄×5 H₂O and 10 ml of hydrochloric acid (37%) in 70 ml of deionized water is slowly metered into the mica suspension at pH 1.8 with stirring. 220 g of a 32% TiCl₄ solution (400 g of TiCl₄/l) are then metered in at a pH of 1.8, during which the pH is kept constant using 32% sodium hydroxide solution. The pH is subsequently raised to 7.5, and 800 ml of sodium water-glass solution (13.0% by weight of SiO₂) are slowly metered in at this pH, during which the pH is kept constant at 7.5 using 10% HCl. After a further stirring time of about 0.5 hour, the pH is lowered to 1.8 using hydrochloric acid (10%), and a solution of 2.3 g of SnCl₄×5 H₂O and 10 ml of hydrochloric acid (32%) in 70 ml of deionized water is metered in. About 200 ml of TiCl₄ solution (400 g/l of TiCl₄) are subsequently metered in slowly at a pH of 1.8. The pH is in each case kept constant using sodium hydroxide solution (32% by weight). After a further post-stirring time of 0.5 hour at pH 1.8, the coated mica pigment is filtered off, washed and dried at 110° C. for 16 hours. Finally, the pigment is calcined at 850° C. for 0.5 hour.

A blue interference pigment having a sharp color change from blue to red on changing from a steep to flat viewing angle is obtained.

### Example 2

100 g of mica having a particle size of 10-60 μm are heated to 80° C. in 2 l of demineralised water with stirring. When this temperature has been reached, a solution consisting of 3 g of SnCl₄×5 H₂O and 10 ml of hydro-chloric acid (37%) in 90 g of water is slowly metered into the mica suspension with vigorous stirring. 220 ml of TiCl₄ solution (400 g of TiCl₄/l) are metered in at a pH of 1.8. The pH is then kept constant at 7.5. The pH is subsequently raised to 7.5, and 830 ml of sodium water-glass solution (13% by weight of SiO₂) are slowly metered in at this pH, during which the pH is kept constant at 7.5 using 10% HCl. A solution of 11.5 g of SnCl₄×5 H₂O and 10 ml of hydrochloric acid (32%) in 350 ml of deionized water is subsequently metered in at pH 1.8. Finally, 220 ml of TiCl₄ solution (400 g of TiCl₄/l) are metered in at pH 1.8. During the addition of the SnCl₄×5 H₂O solutions and TiCl₄ solutions, the pH is in each case kept constant using NaOH solution (32%).

For work-up, the coated mica pigment is filtered off, washed and dried at 110° C. for 16 hours. Finally, the pigment is calcined at 850° C. for 0.5 hour.

A brightly lustrous, green-blue interference pigment whose color is red-violet on viewing at a flat angle is obtained.

### Use Examples

### Example A1

Wet Lip Balm Comprising 10% of Pearlescent Pigment

Phase A

| | | | |
|---|---|---|---|
| Five-layered pigment | Silica, CI77891 (Titanium dioxide), | 10.00% | (1) |
| according to Example 1 | Mica, Tin oxide | | |

Phase B

| | | | |
|---|---|---|---|
| Vegelatum Equiline EU103 | | 11.00% | (2) |
| Candelilla Wax | Candelilla Cera (Candelilla Wax) | 7.00% | (3) |
| Beeswax Bleached | Cera Alba (Beeswax) | 5.00% | (1) |
| Myritol 331 | Cocoglycerides | 8.00% | (4) |
| Ozokerite Wax White # 77W | Ozokerite | 3.00% | (3) |
| Isopropyl Myristate | Isopropyl Myristate | 5.00% | (4) |
| Eusolex ® 2292 | Ethylhexyl Methoxycinnamate, BHT | 7.00% | (1) |
| Eusolex ® OCR | Octocrylene | 4.00% | (1) |
| Oxynex ® K Liquid | PEG-8, Tocopherol, Ascorbyl Palmitate, | 0.10% | (1) |
| | Ascorbic Acid, Citric Acid | | |
| Propyl 4-Hydroxybenzoate | Propylparaben | 0.10% | (1) |
| Color dispersion in castor oil | | 0.50% | |
| Castor Oil | Ricinus Communis (Castor Oil) | 38.80% | (5) |

Phase C

| | | | |
|---|---|---|---|
| Perfume Oil Tendresse # 75418C | Perfume | 0.50% | (6) |

Sources of supply:
(1) Merck KGaA/Rona ®
(2) Natunola health Inc.
(3) Ross Waxes
(4) Cognis GmbH
(5) Henry Lamotte GmbH
(6) Haarmann & Reimer GmbH

Preparation:

The constituents of phase B are heated at 75° C. with stirring until everything has melted. Add phase A and stir well. Then introduce the lipstick composition into the casting apparatus held at 65° C., add phase C and stir for 15 minutes. The homogeneous melt is poured into the casting mould pre-warmed to 55° C.

The moulds are subsequently cooled, and the castings are removed cold. After the lipsticks have been warmed to room temperature, the lipsticks are briefly flame-treated.

### Example A2

Shower gel

Phase A

| | | | |
|---|---|---|---|
| Five-layered pigment | Silica, CI77891 (Titanium dioxide), | 0.10% | (1) |
| according to Example 1 | Mica, Tin oxide | | |
| Keltrol T | Xanthan Gum | 0.75% | (2) |
| Water, Demineralised | Aqua (Water) | 64.95% | |

Phase B

| | | | |
|---|---|---|---|
| Plantacare 2000 UP | Decyl Glucoside | 20.00% | (3) |
| Texapon ASV 50 | Sodium Laureth Sulfate, Sodium | 3.60% | (3) |
| | Laureth-8 Sulfate, Magnesium Laureth | | |
| | Sulfate, Magnesium Laureth-8 Sulfate, | | |
| | Sodium Oleth Sulfate, Magnesium Oleth | | |
| | Sulfate | | |
| Bronidox L | Propylene Glycol, | 0.20% | (3) |
| | 5-Bromo-5-Nitro-1,3-Dioxane | | |
| Perfume Oil Everest 79658 SB | Perfume | 0.05% | (4) |
| (deleted) | | | |
| 1% FD&C Blue No. 1 in Water | Aqua (Water), CI 42090 (FD&C Blue | 0.20% | (5) |
| | No. 1) | | |

Phase C

| | | | |
|---|---|---|---|
| Citric Acid Monohydrate | Citric Acid | 0.15% | (1) |
| Water, Demineralised | Aqua (Water) | 10.00% | |

Sources of supply:
(1) Merck KGaA/Rona ®
(2) Kelco
(3) Cognis GmbH
(4) Haarmann & Reimer GmbH
(5) BASF AG

Preparation:

For phase A, the five-layered pigment is stirred into water. Slowly scatter in the Keltrol T with stirring and stir until it has dissolved. Add phases B and C successively while stirring slowly until everything has distributed homogeneously. Set the pH to from 6.0 to 6.5.

### Example A3

Eyeliner Gel

Phase A

| | | | |
|---|---|---|---|
| Five-layered pigment | Silica, CI77891 (Titanium dioxide), Mica, Tin oxide | 15.00% | (1) |
| according to Example 1 | | | |
| Mica Black | CI 77499 (Iron Oxides), Mica, CI 77891 | 5.00% | (1) |
| | (Titanium Dioxide) | | |
| Ronasphere ® | Silica | 2.00% | (1) |
| Carbopol ETD 2001 | Carbomer | 0.40% | (2) |
| Citric Acid Monohydrate | Citric Acid | 0.00% | (1) |
| Water, Demineralised | | 60.00% | |

Phase B

| | | | |
|---|---|---|---|
| Glycerine, Anhydrous | Glycerine | 4.00% | (1) |
| Triethanolamine extra pure | Triethanolamine | 0.90% | (1) |
| Luviskol VA 64 Powder | PVP/VA Copolymer | 2.00% | (3) |
| Germaben II | Propylene Glycol, Diazolidinyl Urea, | 1.00% | (4) |
| | Methylparaben, Propylparaben | | |
| Water, Demineralised | Aqua (Water) | 9.70% | |

Sources of supply:
(1) Merck KGaA/Rona ®
(2) BF Goodrich
(3) BASF AG
(4) ISP Global Technologies

Preparation:

The five-layered pigment and the Ronasphere® are dispersed in the water of phase A. Acidify using a few drops of citric acid in order to reduce the viscosity, scatter in the Carbopol with stirring. When completely dissolved, slowly stir in the pre-dissolved phase B and adjust the pH to 7.0-7.5.

### Example A4

Eyeshadow

Phase A

| | | | |
|---|---|---|---|
| Five-layered pigment | Silica, CI77891 (Titanium dioxide), | 55.0% | (1) |
| according to Example 2 | Mica, Tin oxide | | |
| Biron ® B 50 | CI 77163 (Bismuth Oxychloride) | 3.00% | (1) |
| Colorona ® Dark Blue | Pearlescent pigment: Mica, | 10.00% | (1) |
| | CI 77891 (Titanium Dioxide), | | |
| | CI 77510 (Ferric Ferrocyanide) | | |
| Magnesium Stearate | Magnesium Stearate | 2.50% | (1) |
| China Clay (deleted) | Kaolin | 5.00% | (1) |
| Hubersorb 600 | Calcium Silicate | 0.50% | (2) |
| Talc | Talc | 11.00% | (1) |

Phase B

| | | | |
|---|---|---|---|
| Amerchol L 101 | Lanolin Alcohol, Paraffinum Liquidum | 10.70% | (3) |
| | (Mineral Oil) | | |
| Super Hartolan | Lanolin Alcohol | 1.00% | (4) |
| Ewalin 1751 | Petrolatum | 1.00% | (5) |
| Propyl 4-Hydroxybenzoate | Propylparaben | 0.10% | (1) |
| Perfume Oil Elegance # 79228 | Perfume | 0.20% | (6) |
| D MD | | | |

Sources of supply:
(1) Merck KGaA/Rona ®
(2) J. M. Huber Corp.
(3) Amerchol
(4) Croda GmbH
(5) H. Erhard Wagner GmbH
(6) Haarmann & Reimer GmbH

Preparation:

Combine and pre-mix the constituents of phase A. Subsequently add the molten phase B dropwise to the powder mixture with stirring. The powders are pressed at 40-50 bar.

### Example A5

Eyeshadow Gel

Phase A

| | | | |
|---|---|---|---|
| Five-layered pigment | Silica, CI77891 (Titanium dioxide), Mica, Tin oxide | 15.00% | (1) |
| according to Example 1 | | | |
| Mica Black | CI 77499 (Iron Oxides), Mica, CI 77891 | 5.00% | (1) |
| | (Titanium Dioxide) | | |
| Ronasphere ® | Silica | 3.00% | (1) |
| Carbopol ETD 2001 | carbomer | 3.00% | (2) |
| Citric Acid Monohydrate | Citric Acid | 0.00% | (1) |
| Water, Demineralised | Aqua (Water) | 60.00% | |

Phase B

| | | | |
|---|---|---|---|
| Glycerine, Anhydrous | Glycerine | 2.00% | (1) |
| Germaben II | Propylene Glycol, Diazolidinyl Urea, | 0.20% | (3) |
| | Methylparaben, Propylparaben | | |
| Triethanolamine extra pure | Triethanolamine | 0.70% | (1) |
| Water, Demineralised | Aqua (Water) | 13.80% | |

Sources of supply:
(1) Merck KGaA
(2) BF Goodrich GmbH
(3) ISP Global Technologies

Preparation:

The five-layered pigment and the Ronasphere® are dispersed in the water of phase A. Acidify using a few drops of citric acid in order to reduce the viscosity, scatter in the Carbopol with stirring. When completely dissolved, slowly stir in the pre-dissolved phase B.

### Example A6

Shimmering Foundation

Phase A

| | | | |
|---|---|---|---|
| Extender W | Mica, CI 77891 (Titanium Dioxide) | 9.00% | (1) |
| Microna ® Matte Yellow | Mica, CI 77492 (Iron Oxides) | 4.00% | (1) |
| Microna ® Matte Red | CI 77491 (Iron Oxides), Mica | 0.40% | (1) |
| Microna ® Matte Black | CI 77499 (Iron Oxides), Mica | 0.30% | (1) |
| Five-layered pigment | Silica, CI77891 (Titanium dioxide), | 4.50% | (1) |
| according to Example 1 | Mica, Tin oxide | | |
| Ronasphere ® | Silica | 5.00% | (1) |

Phase B

| | | | |
|---|---|---|---|
| Blanose 7 HF | Cellulose Gum | 0.20% | (2) |
| Veegum | Magnesium Aluminium Silicate | 1.00% | (3) |
| Texapon K 1296 | Sodium Lauryl Sulfate | 0.60% | (4) |
| Triethanolamine extra pure | Triethanolamine | 0.50% | (1) |
| Titriplex III | Disodium EDTA | 0.25% | (1) |
| Methyl 4-Hydroxybenzoate | Methylparaben | 0.15% | (1) |
| 1,2-Propanediol | Propylene Glycol | 10.90% | (1) |
| Water, Demineralised | Aqua (Water) | 42.95% | |

Phase C

| | | | |
|---|---|---|---|
| Isopropyl Myristate | Isopropyl Myristate | 8.00% | (4) |
| Liquid Paraffin | Paraffinum Liquidum (Mineral Oil) | 3.60% | (1) |
| Crodamol SS | Cetyl Esters | 2.60% | (5) |
| Monomuls 60-35 C | Hydrogenated Palm Glycerides | 1.70% | (4) |
| Stearic Acid | Stearic Acid | 1.50% | (1) |
| Eusolex ® 6300 | 4-Methylbenzylidene Camphor | 1.30% | (1) |
| Eusolex ® 4360 | Benzophenone-3 | 0.50% | (1) |
| RonaCare ™ Tocopherol Acetate | Tocopheryl Acetate | 0.10% | (1) |
| Magnesium Stearate | Magnesium Stearate | 0.10% | (1) |
| Propyl 4-Hydroxybenzoate | Propylparaben | 0.05% | (1) |

Phase D

| | | | |
|---|---|---|---|
| Perfume Oil 200 529 | Perfume | 0.20% | (6) |
| Euxyl K 400 | Phenoxyethanol, Methyldibromo- | 0.20% | (7) |
| | Glutaronitrile | | |

Sources of supply:
(1) Merck KGaA/Rona ®
(2) Aqualon GmbH
(3) Vanderbilt
(4) Cognis GmbH
(5) Croda GmbH
(6) Fragrance Resources
(7) Schülke & Mayr GmbH

Preparation:

Melt all constituents of phase C at about 75° C. and stir until everything has melted. Initially introduce the water of phase B cold, homogenize in the Blanose using the Turrax, scatter in the Veegum, and re-homogenize. Warm to 75° C. and dissolve the other constituents therein with stirring. Stir in the constituents of phase A. Add phase C at 75° C. with stirring and homogenize for 2 minutes. Cool the composition to 40° C. with stirring, add phase D. Cool further to room temperature with stirring and adjust to pH 6.0-6.5 (for example using citric acid solution).

Phase A

| | | | |
|---|---|---|---|
| Five-layered pigment | Silica, CI77891 (Titanium dioxide), | 10.00% | (1) |
| according to Example 2 | Mica, Tin oxide | | |

Phase B

| | | | |
|---|---|---|---|
| Indopol H 100 | Polybutene | 59.90% | (2) |
| Bentone Gel MIO V | Quatemium-18 Hectorite, Propylene | 20.00% | (3) |
| | Carbonate, Paraffinum Liquidum | | |
| | (Mineral Oil) | | |
| Eutanol G | Octyldodecanol | 6.00% | (4) |
| RonaCare ™ Tocopherol Acetate | Tocopheryl Acetate | 1.00% | (1) |
| Dow Corning 1403 Fluid | Dimethiconol, Dimethicone | 3.00% | (5) |
| Propyl 4-Hydroxybenzoate | Propylparaben | 0.05% | (1) |
| Rouge Covapate W 3773 | Ricinus Communis (Castor Oil), | 0.05% | (6) |
| | CI 15850 (D&C) Red No. 6) | | |

Sources of supply:
(1) Merck KGaA/Rona ®
(2) BP Amoco
(3) Elementis Specialities
(4) Cognis GmbH
(5) Dow Corning
(6) Les Colorants Wackherr

Preparation:

All constituents of phase B are weighed out together, heated to 70° C. and stirred well until a homogeneous composition has formed. The pigments are then added and stirred again. The homogeneous mixture is packaged at 50-60° C.

### Example A8

Eyeshadow—Compact Powder

Phase A

| | | | |
|---|---|---|---|
| Five-layered pigment | Silica, CI77891 (Titanium dioxide), | 25.00% | (1) |
| according to Example 1 | Mica, Tin oxide | | |
| Colorona ® Dark Blue | Mica, CI 77891 (Titanium Dioxide), | 5.00% | (1) |
| | CI 77510 (Ferric Ferrocyanide) | | |
| Talc | Talc | 49.50% | (1) |
| Potato Starch | Solanium Tuberosum (Potato Starch) | 7.50% | (2) |
| Magnesium Stearate | Magnesium Stearate | 2.50% | (1) |

Phase B

| | | | |
|---|---|---|---|
| Isopropyl Stearate | Isopropyl Stearate | 9.14% | (3) |
| Cetyl Palmitate | Cetyl Palmitate | 0.53% | (1) |
| Ewalin 1751 | Petrolatum | 0.20% | (4) |
| Perfume Oil Elegance # 79228 | Perfume | 0.20% | (5) |
| D MF | | | |
| Propyl 4-Hydroxybenzoate | Propylparaben | 0.10% | (1) |

Sources of supply:
(1) Merck KGaA
(2) Südstärke GmbH
(3) Cognis GmbH
(4) H. Erhard Wagner GmbH
(5) Haarmann & Reimer GmbH

Preparation:

Combine and pre-mix the constituents of phase A. Subsequently add the molten phase B dropwise to the powder mixture with stirring. The powders are pressed at 40-50 bar.

### Example A9

Loose Eye Powder

Phase A

| | | | |
|---|---|---|---|
| Five-layered pigment | Silica, CI77891 (Titanium | 50.00% | (1) |
| according to Example 1 | dioxide), Mica, Tin oxide | | |

Phase B

| | | | |
|---|---|---|---|
| Micronasphere ® M | Mica, Silica | 5.00% | (1) |
| Talc | Talc | 30.00% | (1) |
| China Clay (deleted) | Kaolin | 8.00% | (1) |
| Magnesium Stearate | Magnesium Stearate | 2.00% | (1) |

Phase C

| | | | |
|---|---|---|---|
| Ceraphyl 368 | Ethylhexyl Palmitate | 4.90% | (2) |
| Propyl 4- | Propylparaben | 0.10% | (1) |
| hydroxybenzoate | | | |

Sources of supply:
(1) Merck KGaA/Rona ®
(2) ISP Global Technologies

Preparation:

All constituents of phase B are weighed out together, mixed well and sieved at 100 μm. Subsequently add phase A and mix again. Phase C is added dropwise to the composition with stirring.

### Example A10

Cream Mascara (O/W)

Phase A

| | | | |
|---|---|---|---|
| Mica Black | CI 77499 (Iron Oxides), Mica, CI 77891 | 10.00% | (1) |
| | (Titanium Dioxide) | | |
| Five-layered pigment | Silica, CI77891 (Titanium dioxide), | 5.00% | (1) |
| according to Example 2 | Mica, Tin oxide | | |

Phase B

| | | | |
|---|---|---|---|
| Stearic Acid | Stearic Acid | 8.00% | (1) |
| Beeswax, Bleached | Cera Alba (Beeswax) | 6.00% | (1) |
| Carnauba Wax 2442 L | Copernicia Cerifera (Carnauba Wax) | 4.00% | (2) |
| Eutanol G | Octyldodecanol | 3.00% | (3) |
| Arlacel 83 V | Sorbitan Sesquioleate | 2.00% | (4) |
| Propyl 4-Hydroxybenzoate | Propylparaben | 0.10% | (1) |
| RonaCare ™ Tocopherol Acetate | Tocopheryl Acetate | 0.50% | (1) |

Phase C

| | | | |
|---|---|---|---|
| Water, Demineralised | Aqua (Water) | 50.84% | |
| Triethanolamine extra pure | Triethanolamine | 2.30% | (1) |
| Water Soluble Shellac SSB 63 | Shellac | 8.00% | (5) |
| Methyl 4-Hydroxybenzoate | Methylparaben | 0.25% | (1) |
| RonaCare ™ Biotin | Biotin | 0.01% | (1) |

Sources of supply:
(1) Merck KGaA/Rona ®
(2) Kahl & Co.
(3) Cognis GmbH
(4) Uniqema
(5) Paroxite Ltd.

Preparation:

Melt all constituents of phase B together at about 80° C., stir until everything has melted. Stir in the effect pigments of phase A. Dissolve the shellac of phase C in the water, warm to 75° C. Add the remaining constituents of phase C, dissolve. Slowly add phase C to phase A/B at 75° C. with stirring, homogenize for 2 minutes. Cool the composition to room temperature with stirring.

### Example A11

Nail Varnish

Phase A

| | | | |
|---|---|---|---|
| Five-layered pigment | Silica, CI77891 (Titanium dioxide), | 2.00% | (1) |
| according to Example 1 | Mica, Tin oxide | | |
| Thixotropic Nail Varnish Base | Toluene, Ethyl Acetate, Butyl Acetate, | 98.00% | (2) |
| 1348 | Nitrocellulose, Tosylamide/Formaldehyde | | |
| | Resin, Dibutyl Phthalate, Isopropyl | | |
| | Alcohol, Stearalkonium Hectorite, | | |
| | Camphor, Acrylate Copolymer, | | |
| | Benzophenone-1 | | |
| Color Dispersion with | | | |
| Nitrocellulose Lacquer (q.s.) | | | |

Sources of supply:
(1) Merck KGaA/Rona ®
(2) International lacquers S. A.

Preparation:

The five-layered pigment is weighed out together with the varnish base, mixed well by hand using a spatula and subsequently stirred at 1000 rpm for 10 minutes.

### Example A12

Shampoo

Phase A

| | | | |
|---|---|---|---|
| Five-layered pigment | Silica, CI77891 (Titanium dioxide), | 0.05% | (1) |
| according to Example 2 | Mica, Tin oxide | | |
| Carbopol ETD 2020 | Acrylate/C10-30 Alkyl Acrylate | 0.90% | (2) |
| | Crosspolymer | | |
| Water, Demineralised | Aqua (Water) | 59.90% | |

Phase B

| | | | |
|---|---|---|---|
| Triethanolamine extra pure | Triethanolamine | 0.90% | (1) |
| Water, Demineralised | Aqua (Water) | 10.00% | |

Phase C

| | | | |
|---|---|---|---|
| Plantacare 2000 UP | Decyl Glucoside | 20.00% | (3) |
| Texapon ASV | Magnesium Oleth Sulfate, Sodium | 8.00% | (3) |
| | Oleth Sulfate, Magnesium | | |
| | Laureth-8 Sulfate, Sodium | | |
| | Laureth-8 Sulfate, Magnesium | | |
| | Laureth Sulfate, Sodium Laureth Sulfate | | |
| Bronidox L | Propylene Glycol, | 0.20% | (3) |
| | 5-Bromo-5-Nitro-1;3-Dioxane | | |
| Perfume Oil Everest 79658 SB | Perfume | 0.05% | (4) |

Sources of supply:
(1) Merck KGaA
(2) BF Goodrich GmbH
(3) Cognis GmbH
(4) Haarmann & Reimer GmbH

Preparation:

For phase A, stir the five-layered pigment into the water. Acidify using a few drops of citric acid (10%) in order to reduce the viscosity, and slowly scatter in the Carbopol with stirring. When completely dissolved, slowly add phase B. The constituents of phase C are then added one after the other.

### Example A13

Sparkling Body Cream (O/W)

Phase A

| | | | |
|---|---|---|---|
| Five-layered pigment | Silica, CI77891 (Titanium | 3.00% | (1) |
| according to Example 2 | dioxide), Mica, Tin oxide | | |
| Carbopol ETD 2001 | Carbomer | 0.60% | (2) |
| Citric Acid Monohydrate | Citric Acid | | (1) |
| Water, Demineralised | Aqua (Water) | 40.00% | |

Phase B

| | | | |
|---|---|---|---|
| RonaCare ™ Allantoin | Allantoin | 0.20% | (1) |
| 1,2-Propanediol | Propylene Glycol | 3.00% | (1) |
| Euxyl K 400 | Phenoxyethanol, | 0.10% | (3) |
| | Methyldibromo- | | |
| | Glutaronitrile | | |
| Chemag 2000 | Imidazolidinyl Urea | 0.30% | (4) |
| Methyl 4-Hydroxybenzoate | Methylparaben | 0.15% | (1) |
| Water, Demineralised | Aqua (Water) | 27.65% | |

Phase C

| | | | |
|---|---|---|---|
| Hostaphat KL 340 N | Dilaureth-4 Phosphate | 3.00% | (5) |
| Cetyl Alcohol | Cetyl Alcohol | 2.00% | (1) |
| Liquid Paraffin | Paraffinum Liquidum | 10.00% | (1) |
| | (Mineral Oil) | | |
| Cetiol V | Decyl Oleate | 6.00% | (6) |
| Propyl 4-Hydroxybenzoate | Propylparaben | 0.05% | (1) |

Phase D

| | | | |
|---|---|---|---|
| Triethanolamine | Triethanolamine | 0.35% | (1) |
| Water, Demineralised | Aqua (Water) | 3.50% | |

Phase E

| | | | |
|---|---|---|---|
| Perfume Oil 72979 | Perfume | 0.10% | (7) |

Sources of supply:
(1) Merck KGaA
(2) BF Goodrich GmbH
(3) Schülke & Mayr GmbH
(4) Chemag AG
(5) Clariant GmbH
(6) Cognis GmbH
(7) Haarmann & Reimer GmbH

Preparation:

Disperse the five-layered pigment in the water of phase A. If necessary, acidify using a few drops of citric acid in order to reduce the viscosity. Scatter in the Carbopol with stirring. When completely dissolved, slowly stir in the pre-dissolved phase B. Heat phase A/B and phase C to 80° C., stir phase C into phase A/B, homogenize, neutralise using phase D, homogenize again and cool with stirring. Add the perfume oil at 40° C., cool to room temperature with stirring.

### Example A14

Plastic

In each case,

a) 1% of pigment from Example 1

b) 1% of pigment from Example 2

c) 1% of pigment from Example 1 and 0.1% of PV True Blue B2G01 (Pigment Blue 15.3)

are added to polypropylene PP Stamylan PPH10 (DSM) or polystyrene 143E (BASF) plastic granules.

The pigmented granules are subsequently converted into stepped plates in an injection-moulding machine.

### Example A15

Printing Ink

The pigment is stirred into the solvent-containing binder at 600 rpm, and the printing inks are subsequently knife-coated onto black/white cards.

Ink No. 1:

| | |
|---|---|
| 88.0 g | of Gebr. Schmidt 95 MB 011 TW |
| 10.0 g | of pigment from Example 1 |
| 2.0 g | of Gebr. Schmidt 95 MB 022-TW (green) |

Ink No. 2:

| | |
|---|---|
| 88.0 g | of Gebr. Schmidt 95 MB 011 TW |
| 10.0 g | of pigment from Example 2 |
| 2.0 g | of Gebr. Schmidt 95 MB 022-TW (green) |

### Example A16

Automotive Paint

| | |
|---|---|
| 2.00 g | of pigment from Example 1 |
| 1.50 g | of Heliogen Blue L 6930 |
| 0.20 g | of Hostaperm Green 8G |
| 0.05 g | of carbon black FW 200 |
| 66.60 g | of basecoat (A4) MP system (solids content = 19%) |
| 29.65 g | of thinner mixture |

The entire disclosure[s] of all applications, patents and publications, cited herein and of corresponding German application No. 10229256.6, filed Jun. 28, 2002 and German-application No. 10251378.3, filed Nov. 1, 2002 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A pigment comprising a platelet-shaped substrate comprising thereon at least a 5-layer sequence of
(A) a layer of SnO₂, having a layer thickness of 0.1-50 nm,
(B) a layer of TiO₂ in rutile modification, having a layer thickness of 10-800 nm,
(C) a colorless coating having a refractive index n≦1.8,. having a layer thickness of 20-800 nm,
(D) a layer of SnO₂, having a layer thickness of 0.1-50 nm,
(E) a layer of TiO₂ in the rutile modification, having a layer thickness of 10-800 nm
and optionally
(F) an outer protective layer.

2. The five-layered pigment according to claim 1, wherein the platelet-shaped substrate is natural or synthetic mica, glass, or Al₂O₃, SiO₂ or TiO₂ platelets.

3. The five-layered pigment according to claim 1, wherein layer (C) is SiO₂, Al₂O₃, AlO(OH), B₂O₃, MgF₂, MgSiO₃ or a mixture thereof.

4. The five-layered pigment according to claim 1, having an outer protective layer (F) increasing light, temperature and weather stability.

5. A process for the preparation of a five-layered pigment according to claim 1, comprising coating the substrates by a wet-chemical method with hydrolytic decomposition of metal salts in aqueous medium.

6. In a paint, coating, powder coating, printing ink, security printing ink, plastic, ceramic material, glass, dopants for laser marking of paper or plastic, cosmetic formulation, pigment preparation or dry pigment preparation comprising a luster or effect pigment, the improvement wherein the luster or effect pigment is a five-layered pigment according to claim 1.

7. A pigment preparation comprising at least one binder, and at least one five-layered pigment according to claim 1.

8. A Pigment preparation according to claim 7, which is a dry preparation.

9. The dry preparation according to claim 8, comprising from 0 to 8% by weight of water and/or a solvent or solvent mixture.

10. The dry preparation according to claim 9, in the form of pellets, granules, chips or briquettes.

11. A hydrophilic, hydrophobic or lipophilic cosmetic formulation comprising at least one five-layered pigment according to claim 1.
